(19) United States
(12) Patent Application Publication
(10) Pub. No.: US 2007/0162095 A1
(43) Pub. Date: Jul. 12, 2007

(54) Modular visualization stylet apparatus and methods of use

(75) Inventors: Zebadiah Kimmel, Brookline, MA (US); Raymond Glassenberg, Wilmette, IL (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: EZC Medical LLC, San Francisco, CA (US)

(21) Appl. No.: 11/326,879

(22) Filed: Jan. 6, 2006

(51) Int. Cl. A61F 2/00

(52) U.S. Cl. 607/102

(57) ABSTRACT

Visualization stylets and methods of use are provided, in which the visualization stylets include modular components that allow interchangeability of imaging devices and lenses, and the use of forward-facing or lateral-facing lens orientations. Optionally, the lens may be focused remotely. A reduced insertion profile is provided by configuring the circuitry of the imaging device so that it is disposed substantially perpendicular to a plane of a pixel array of the imaging device.

### FIELD OF THE INVENTION

The present invention relates to visualization apparatus, and in particular to stylets having modular features allowing for rapid customization and modification to suit a clinician's needs.

### BACKGROUND OF THE INVENTION

Proper treatment and diagnosis of a patient often involves a thorough examination. In conducting an examination, clinicians often use visualization devices to probe ducts, orifices, bodily openings, or other spaces. One such device is a visualization stylet, typically a long thin probe that employs optical fibers to transmit images of interior bodily structures. Previously-known visualization stylet designs suffer numerous disadvantages.

Typically optical fibers are used to transmit illumination and images. For example, U.S. Pat. No. 5,394,865 to Salerno describes a visualization stylet that utilizes fiber optic cables. This stylet is designed to be reused and sanitized in an autoclave. Such sterilization procedures are time consuming and expensive. Accordingly, it is desirable to provide a stylet that does not require sterilization by autoclave after use.

Other previously-known medical imaging device designs utilize an imaging device, such as a CCD or CMOS, to gather images. For example, U.S. Pat. No. 6,117,071 to Ito, et al. describes an endoscope having a CCD located in an imaging unit near its distal end to gather images. In addition to requiring sterilization after each use, the device described in Ito also has a relatively large insertion profile, i.e., cross sectional area, thereby limiting its use to openings of sufficient size. Accordingly, it would be desirable to provide a stylet having a relatively small insertion profile.

Other previously-known visualization stylets employ optics having a fixed focal length. Other stylet designs provide mechanisms for focusing, but with increased insertion profile. Accordingly, it would be desirable to provide a stylet offering of a range of focal lengths, but without the additional cost and complexity attendant upon use of focusing systems that significantly increase the insertion profile.

### SUMMARY OF THE INVENTION

In view of the above-listed disadvantages with the prior art, it is an object of the present invention to provide a visualization stylet that does not require sterilization by autoclave after use.

It is another object of the present invention to provide a visualization stylet having a relatively small insertion profile.

It is a further object of the present invention to provide to provide a visualization stylet that offers a range of focal lengths, but without focusing systems.

These and other advantages are accomplished by providing a visualization stylet having a variety of single-use modular components that provide versatility by offering a selection of lenses and/or imaging devices. Accordingly, when using the visualization stylet for a particular patient, a clinician may first select a forward-facing imaging device and a lens with a wide range of view. The clinician then may remove and replace the lens with another lens capable of greater magnification. Later, the clinician may remove and replace the forward-facing imaging device with a lateral imaging device for additional examination. Finally, at the conclusion of the examination, the clinician may dispose of each modular component that has been inserted into the patient, while preserving a reusable external unit.

To avoid unnecessary material cost and to preserve storage space, individual modules of the stylet may be sterilized and packaged separately in sterile containers. A clinician need select only the modules intended to be used at a particular time, avoiding unnecessary waste of resources.

In use, a distal portion of the apparatus containing the image gathering device is inserted into the patient. In this specification, the terms “distal” and “proximal” refer to the perspective of the clinician or other user. The reusable external unit may be connected to a monitor, television, or other output device that allows the clinician to see the images gathered by the image gathering device in real-time. The reusable external unit also may contain a power source, such as a battery, and controls, such as an on/off switch that activates features on the attached module.

The imaging device preferably is a complementary metal oxide semiconductor (“CMOS”), and more preferably is a CMOS with analog output. The insertion profile of the stylet may be further reduced by providing an imaging device coupled to an elongated circuitry board, as opposed to previously-known square configurations in which the imaging device is centered and surrounded by circuitry. In the visualization stylet of the present invention, the circuit may be disposed on a relatively rigid surface, e.g., a circuit board substrate, or may be disposed on a flexible printed circuit board, e.g., formed by thin film deposition on a polymer substrate.

Illumination devices also may be incorporated into the visualization stylet to illuminate the area being imaged. Examples of suitable illumination devices include light emitting diodes (LEDs) and infrared lights. In a preferred embodiment, the illumination device is configured as an annulus disposed concentrically around the imaging device. Preferably the illumination device is located in the same module as the imaging device, and any additional lens modules may include light-transmissive material to project the light rays in a desired direction. Alternatively, the illumination device may be located in a lens module rather than in an imaging module.

The stylet of the present invention also may include a module having an imaging device and a lens capable of variable focus, thereby allowing a range of focal lengths without necessitating the removal of the stylet from the patient.

The insertion profile may be further reduced by utilizing the metallic wires, used to transmit electrical signals to the illumination device and imaging device, to retain the shape of the visualization stylet.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIG. 1 is a perspective view of an illustrative embodiment of a visualization stylet incorporating features of the present invention;

FIG. 2 is a perspective view of the proximal portion of the visualization stylet of FIG. 1;

FIG. 3 is a perspective view of the distal portion of the visualization stylet of FIG. 1;

FIG. 4 is a cross sectional view of a proximal portion of a visualization stylet of the present invention;

FIG. 5 is a cross sectional view of a distal portion of a visualization stylet of the present invention;

FIGS. 6A-C are cross sectional views of embodiments of lens modules for use with the visualization stylet of the present invention;

FIG. 7 depicts a perspective view of an imaging device suitable for use in the present invention;

FIG. 8 depicts a cross sectional view of an alternative embodiment of an imaging module for use with the visualization stylet of the present invention; and

FIGS. 9A and 9B depict cross sectional views of another alternative embodiment of an imaging module for use in the visualization stylet of the present invention.

### DETAILED DESCRIPTION OF THE INVENTION

The present invention is directed to a visualization stylet having modular components and other features that enhance usability and reduce the insertion profile of the device. These features allow a clinician to select a desired combination of an imaging device and lens configuration from amongst an assortment of available components. Following use, the modular components that have been inserted into a patient or otherwise contaminated may be disposed, while unused components and external components remain available for future use.

Referring to FIG. 1, a preferred embodiment of the visualization stylet of the present invention is described. Device 10 includes external controller 11, extension module 12, imaging module 13, lens module 14, and conduit 15 having connector 16. Operation of device 10 is controlled using external controller 11, which preferably comprises housing 17 formed of a rigid or semi-rigid material such metal, ceramic, or plastic, although other materials also may be acceptable. Power switch 18, optional secondary switch (not.shown), battery cover 19, and optional clasps 20 are mounted on housing 17. Although depicted as having a cylindrical shape, external controller 11 may be available in different configurations, such as a pistol-grip.

Conduit 15 extends from external controller 11 and terminates in connector 16. Connector 16 may be coupled to receiving connector 21, which communicates with viewing screen 22. Conduit 15 preferably comprises a wire, cable, or other medium for transmitting electrical signals, whereas connector may be an RCA jack, RCA plug, or similar device that preferably allows rapid connection.

Extension module 12 comprises an elongated shaft having distal end 23 and proximal end 24. Extension module 12 may be provided in a variety of lengths, and may be configured to attach to other extension modules 12, allowing further increases in length. Proximal end 24 is attachable to external unit 11, and is secured by optional clasps 20. Distal end 23 is configured to attach to imaging module 13 so as to avoid significant discontinuities along the outer surface of device 10. Extension module 12 preferably comprises a pliable material, such as a polymer, that allows extension module 12 to be bent or configured as required by the clinician or other user to fit the anatomy of a specific patient. In other embodiments, extension module 12 may be rigid or flexible, and may contain jointed or maneuverable segments.

Imaging module 13 has distal end 25 and proximal end 26. Distal end 25 is configured to attach to lens module 14, whereas proximal end 26 is configured to attach to extension module 12. In some embodiments, imaging module 13 may comprise a relatively flexible or pliable exterior, whereas in other embodiments imaging module 13 may have a less flexible exterior. Lens module 14 also may comprise one or more lenses and therefore need not be configured to attach to a separate lens module.

Lens module 14 is disposed at the distal end of device 10 and has distal end 27 and proximal end 28. Distal end 27 is configured to allow light rays to enter device 10, whereas proximal end 28 is configured to mate with imaging module 13 without a significant discontinuity along the outer surface of the device. Preferably, lens module 14 is relatively short and has a less flexible exterior. Lens module 14 preferably comprises a light-transmissive component allowing light to be directed in a distal direction. This feature allows lens module 14 to transmit light that is generated from within imaging module 13 to a point distal to device 10. In other embodiments, lens module 14 may contain a light source, such as an LED, that receives power via an electronic coupling between the lens module and the imaging module 13.

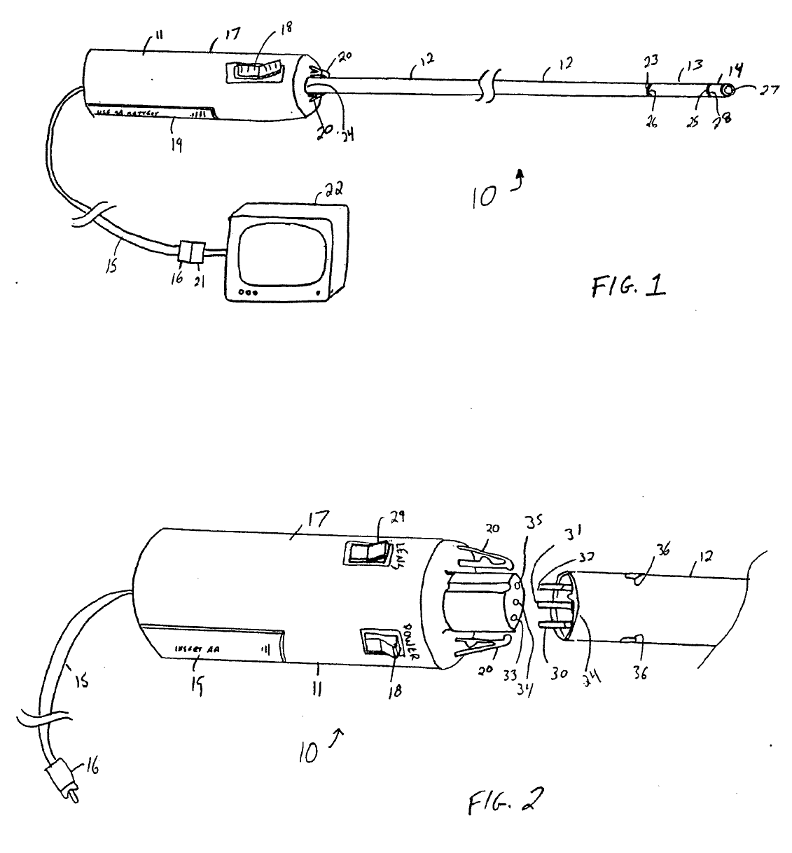

Referring now to FIG. 2, external controller 11 is described in greater detail disconnected from extension module 12, and having optional secondary switch 29. Connectors 30, 31, and 32 extend from extension module 12 toward external controller 11. Connector 30 couples to connector 33 to transmit power to module 13. Connector 31 couples to connector 34 to receive signals from the imaging module 13. Optional connector 32 couples to connector 35 to communicate power or signals from optional secondary switch 29. Although connectors 30, 31, and 32 are depicted as male connection members extending from extension module 12, other connectors and configurations known in the art such as screw threads may be used. Additionally, other embodiments may include a connection to supply ground voltage.

Still referring to FIG. 2, the proximal end 24 of extension module 12 includes indentions 36 configured to engage clasps 20 to reduce the risk of unintended detachment of extension module 12 from external controller 11. Extension module 12 is attached to external controller 11 by sliding connectors 30, 31, and 32 into corresponding connectors 33, 34, and 35, respectively. Once connectors 30, 31, and 32 are fully engaged with the respective connectors 33, 34, and 35, optional clasps 20 engage with optional indentations 36. Extension module 12 later may be released by actuating clasps 20 to disengage indentations 36, and disengaging connectors 30, 31, and 32 from the respective connectors 33, 34, and 35. It will be understood that other attachment assemblies are known in the art and are intended to be included within the scope of the present invention.

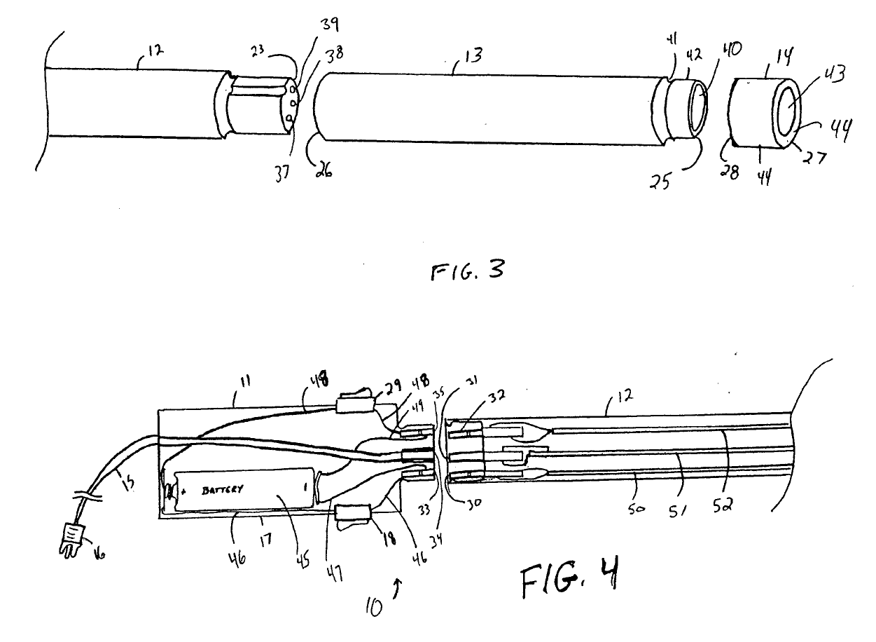

In FIG. 3, distal end 23 of extension module 12 is shown disconnected from imaging module 13, which in turn is disconnected from lens module 14. Distal end 23 of extension module 12 has connectors 37, 38, and 39 configured to engage connectors (not shown) near distal end 26 of imaging module 13. Imaging module 13 includes one or more connectors that engage one or more or connectors 37, 38, and 39. Preferably, connectors 37, 38, and 39 also are configured in the same manner as connectors 33, 34, and 35, such that distal end 23 of a first extension module 12 may connect to the proximal end of a second extension module, thereby allowing device 10 to be lengthened.

Distal end 25 of imaging module 13 has opening a 40 that allows light rays to enter the component. Light rays pass through lens module 14 prior to entering imaging module 13, as discussed in further detail below. Imaging module preferably includes groove 41 and narrowed section 42 configured to securely couple lens module 14 with imaging module 13. Lens module 14 includes lens 43 that directs visible light, infrared light, or other light toward imaging module 13. In a preferred embodiment, lens module 14 comprises exterior 44 that is light-transmissive.

Referring now to FIG. 4, the interior of external controller 11 and proximal end 24 of extension module 12 are described. Conduit 15 is coupled via connector 34 to viewing screen 22 (see FIG. 1). Electrical power from power source 45, such as a battery or rechargeable battery, is communicated via connector 33, conduits 46 and 47, and switch 18 to imaging module 13. Power source 45 also optionally may communicate via connector 35 and optional conduits 48 and 49 to imaging module 13 under control of optional secondary switch 29. In other embodiments, power source 45 is external to external controller 11, such as an external A/C outlet connected to device 10 via an electrical connector and an A/C adapter.

Conduits 50, 51, and 52 are disposed in extension module 12 and are configured to couple to connectors 30, 31, and 32, respectively. Conduits 50, 51, and 52 also are in communication with connectors 37, 38, and 39, respectively, at distal end 23.

One or more of conduits 50, 51, and 52 preferably comprises a malleable material, such as copper wire, that enables extendable module 12 to be selectively bent, curved, angled, or otherwise have a shape impressed upon them by a clinician with relative ease. In this manner, extension module 12 may be configured without the need for a separate malleable interior component, thereby reducing the number of components within extension module 12 and allowing for a reduced insertion profile.

Referring now to FIG. 5, further details of imaging module 13 and lens module 14 are described. Connectors 53 and 54 connect to connectors 37 and 38, respectively. Power is communicated to imaging device 57 from connector 53 via conduit 55. Imaging signals are communicated from imaging device 57 to connector 54 via conduit 56.

Light source 58 receives power via conduit 59, which may attach to imaging device 57. Light source 58 preferably comprises one or more LEDs or other illumination sources. More preferably, light source 58 is configured as an annulus disposed near distal end 25 and directing light in a distal direction.

Imaging module 13 also comprises ridge 60 and inset 61 configured to couple with groove 41 and narrowed section 42 of extension module 12 to secure the modules together. Likewise, imaging module 13 comprises groove 62 and narrowed section 63 configured to couple with ridge 64 and inset 65 of lens module 14. Other simple mechanical connection mechanisms may be employed.

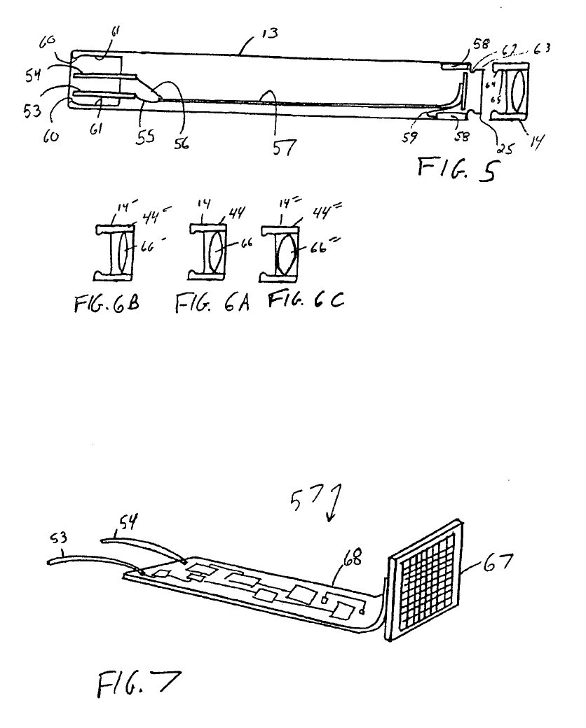

With respect to FIGS. 6, several embodiments of lens modules are described. In FIG. 6A, lens module 14 comprises lens 66 and exterior 44. Exterior 44 preferably is light-transmissive and is configured to direct light emitted from light source 58 in a distal direction. Accordingly, lens module 14 may transmit light to an area to be viewed by imaging device 57, without need for separate electrical connectors to lens module 14. In some embodiments, lens module 14 may contain a light source that is in electrical communication with imaging module 13 via electrical connectors.

In FIG. 6B, lens module 14′ comprises lens 66′ and exterior 44′, and in FIG. 6C lens module 14″ comprises lens 66″ and exterior 44″. Each numbered component having a prime (′) or double prime (″) is described similarly as the like component having no prime designator. In accordance with one part of the present invention, lenses 66, 66′, and 66″ have different optical characteristics. For example, lens 66′ may have less magnification than lens 66, whereas lens 66″ may have greater magnification than lens 66. One or more lenses 66, 66′, or 66″ may be filtered, polarized, or possess other optical properties desirable for a specific application.

In use, a clinician may attach a lens module 14, 14′, or 14″ to imaging module 13 just prior to examining a patient. During the examination process, the clinician may wish to increase or decrease the magnification, and may remove device 10, replace the lens module with one having the desired optical characteristics, and then resume the examination.

With respect to FIG. 7, imaging device 57 preferably comprises a CMOS chip, and more preferably comprises a CMOS chip with analog output that can directly interface with video hardware using NTSC/PAL format. CMOS chips with analog output capable of directly interfacing with video hardware using NTSC/PAL format are commercially available, such as models OV7940 and OV7941 available through OmniVision Technologies, Inc., of Sunnyvale, Calif. Having direct analog output in the fashion described averts the need for additional circuitry for converting digital image signals into analog image signals. In other embodiments, a chip of standard configuration may be utilized.

Unlike previously-known CMOS chips, imaging device 57 preferably is configured to reduce the insertion profile of device 10. In particular, imaging device 57 may be configured with pixel array 67 disposed substantially perpendicular to the plane of imaging circuitry 68. Generally, CMOS chips are fabricated with the imaging circuitry surrounding the pixel array. This configuration is useful in many large-scale applications, but presents significant drawbacks when attempting to incorporate CMOS technology in small scale applications, as with certain imaging devices used in the field of medicine. In accordance with one aspect of the present invention, image device 57 is configured with circuitry 68 disposed in an asymmetric, elongated manner as opposed to a conventional square orientation surrounding the pixel array 67. Circuitry 68 may be disposed on a relatively rigid circuit board, or more preferably may be disposed on a printed circuit board formed on a flexible polymer material.

Circuitry 68 preferably provides analog output readable by hardware using NTSC/PAL technology. In this manner, circuitry 68 may omit analog-to-digital converter circuitry and thereby reduce the number of required components. Imaging device 57 further may be reduced in size by omitting the infrared filter commonly employed with CMOS chips.

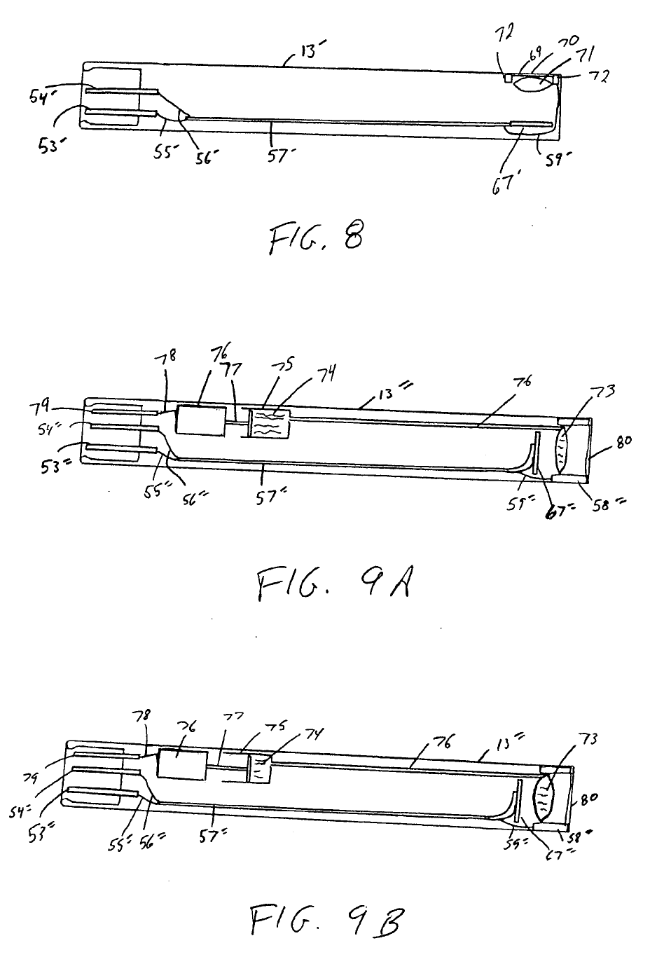

Referring to FIG. 8, an alternative embodiment of imaging module 13′ comprises imaging device 57′ having laterally orientated pixel array 67′. Opening 69 permits light to enter through the lateral exterior surface of imaging device 13′, and preferably includes transparent cover 70 that permits light rays to pass, but prevents fluids and/or particles from entering module 13′. Light source 72 is powered via conduit 59′ and preferably comprises one or more LEDs. Connectors 54′ and 53′ communicate with imaging device 57′ via 55′ and 56′. Imaging module 13′need not connect to a separate lens module, since lens 71 is incorporated directly into imaging module 13′.

In FIGS. 9, an embodiment of an imaging module having a variable-focus lens is described. Imaging module 13″ is similar in design to imaging modules 13 and 13′, but further comprises flexible lens 73. In other embodiments, the lens may be a rigid lens that may be focused by moving the lens forward or backward along a track or by other mechanical means.

Flexible lens 73 comprises a translucent sac filled with fluid 74. The sac is in fluid communication with reservoir 75 via conduit 76, so that the optical properties of the lens may be controlled by varying the volume of fluid within the sac. The volume of reservoir 75 may be selectively altered using pump 76 and piston 77. Pump 76 receives power signals via conduit 78 connected to connector 79, which is configured to engage connector 39. Optional secondary switch 29 may be configured to control operation of pump 76. In use, a clinician wishing to alter the optical characteristics of lens 73 may activate secondary switch 29, to cause piston 77 to displace fluid 74 from reservoir 75 and into lens 73. FIG. 9A depicts imaging module 13″ with an initial distribution of fluid 74 between lens 73 and reservoir 75. FIG. 9B depicts a different moment in which piston 77 has displaced an amount of fluid 74 from reservoir 75 and into lens 73, thereby enhancing the magnification of lens 73. If piston 77 then is retracted by reversing pump 76, e.g., by moving secondary switch 29 to a second position, fluid 74 is drawn from lens 73 and into reservoir 75, so that lens 73 returns to the configuration depicted in FIG. 9A.

As in the preceding embodiments, light source 58″ transmits light in a distal direction. In a preferred embodiment, shield 80 is disposed over the distal opening of imaging module 13″ to prevent foreign matter from contacting lens 73. In other embodiments, shield 80 is not necessary, as the lens may be exposed to the environment.

It should be understood that while imaging module 13″ is depicted as a forward-facing device, capable of capturing a forward-looking image, the same principles may be applied to form a laterally-viewing imaging module with a flexible lens.

Combinations of the concepts presented here may also be prepared. For example, a device may be constructed having an image module with a flexible exterior, an imaging device with circuitry on a flexible printed circuit board, and a flexible lens. The foregoing embodiments are meant to be exemplary and in no way limit the scope of the present invention.

A preferred method of using device 10 of FIG. 1 is now described, for example to internally examine a patient. A clinician first assembles device 10 by . selecting external controller 11, extension module 12 of an appropriate length, forward-facing imaging module 13, and a lens module having a wide angle lens. It should be noted that the extension module 12 is optional, and imaging module 13 otherwise may be attached directly to external controller 11. Extension module 12 is aligned and connected to external controller 11 and imaging module 13. Lens module 14, 14′ or 14″ is connected to distal end 25 of imaging module 13 and conduit 15 is coupled to viewing screen 22 via connector 16.

Switch 18 then is activated to provide power to light source 58 and imaging device 57. Data from imaging device 57 is transmitted to viewing screen 22, allowing the clinician to visualize images distal to device 10. The clinician may bend extension module 12 to a desired shape to facilitate insertion of the device.

Device 10 then is inserted into the patient with the clinician monitoring the progress of the insertion by observing viewing screen 22. Once in place, the clinician may locate and examine a desired area or organ. If, for example, the clinician desires greater magnification, device 10 may be removed from the patient, the lens module may be detached and replaced with another lens module having greater magnification, and the clinician may reinsert device 10 to examine the desired area in greater detail.

The clinician also may desire to examine a target region within the patient from a different perspective. Accordingly, the clinician may remove device 10, disengage the imaging module 13 from the extension module, and attach imaging module 13′ that provides lateral-viewing capabilities. The clinician then may re-insert the device and continue the examination. At the conclusion of the examination, the clinician may disconnect extension module 12 from external controller 11 and discard the used modular components, while retaining the external controller for future use.

It is believed that the operation and construction of the present invention will be apparent from the foregoing description and, while the invention shown and described herein has been characterized as particular embodiments, changes and modifications may be made therein without departing from the spirit and scope of the invention as defined in the following claims.

What is claimed is:

1. A visualization device comprising:
an external controller;
an extension module having a distal end and a proximal end, the proximal end of the extension module configured to be removably attached to the external controller;
an imaging module including an imaging device disposed in communication with the external controller, the imaging module having a distal end and a proximal end configured to be removably attached either to the distal end of the extension module or to the distal end of the external controller; and
a lens module having a viewing surface and a proximal end configured to be removably attached to the distal end of the imaging module.

2. The device of claim 1 further comprising a light source disposed within the imaging module.

3. The device of claim 2 wherein the light source is configured as an annulus.

4. The device of claim 3 wherein the imaging device is a CMOS device configured to output an analog signal.

5. The device of claim 4 wherein the external controller further comprises a conduit adapted to couple with a visualization device.

6. The device of claim 5 wherein the CMOS device is in communication with the visualization device.

7. The device of claim 6 wherein the CMOS device comprises a pixel array and circuitry, the circuitry disposed along an axis substantially perpendicular to the pixel array.

8. The device of claim 7 wherein the imaging module communicates with the external controller through conduits disposed within the extension module, the conduits within the extension module configured to retain an impressed shape.

9. The device of claim 8 wherein the external module further comprises a switch in communication with the power source.

10. A visualization device comprising:
an external controller;
an extension module having a distal end and a proximal end, the proximal end of the extension module configured to be removably attached to the external controller;
an imaging module having a distal end and a proximal end configured to be removably attached either to the extension module or to the external controller;
a imaging device disposed within the imaging module and configured to selectively communicate with the external controller;
a light source disposed within the imaging module; and
a lens disposed within the imaging module.

11. The device of claim 10 wherein the lens comprises a fluid-filled sac.

12. The device of claim 11 further comprising a reservoir in fluid communication with the interior of the lens.

13. The device of claim 12 further comprising a pump in communication with the reservoir, the pump configured to selectively move fluid between the reservoir and the sac.

14. The device of claim 10 wherein the lens is configured to selectively focus light rays onto the imaging device.

15. The device of claim 10 wherein the lens is configured to direct light rays from a lateral location onto the imaging device.

16. The device of claim 10 wherein the light source is annular.

17. The device of claim 16 wherein the imaging device is a CMOS device configured to output an analog signal.

18. The device of claim 17 wherein the external controller further comprises a conduit adapted to couple with a visualization device and CMOS device is in communication with the visualization device.

19. An imaging module for use with a visualization device comprising:
a housing having a distal end and a proximal end; and
a CMOS device having a pixel array and circuitry, wherein the CMOS device is disposed within the housing and the circuitry is disposed substantially perpendicular to a plane of the pixel array; and
wherein the imaging.module is disposable.

20. A method of examining an interior space comprising:
providing a device comprising an external controller coupled to an extension module, an imaging module having a imaging device and a light source, and a lens;
attaching the conduit to a visualization device;
providing power to the light source and imaging device;
inserting the imaging module into the interior space;
observing the visualization device;
removing the imaging module from the interior space; and
discarding the imaging module.
